(19) United States
(12) Patent Application Publication
(10) Pub. No.: US 2020/0010326 A1
(43) Pub. Date: Jan. 9, 2020

(54) INTERCALATION AGENT FOR RAPID GRAPHITE EXFOLIATION IN MASS PRODUCTION OF HIGH-QUALITY GRAPHENE

(71) Applicant: GUANGZHOU SPECIAL PRESSURE EQUIPMENT INSPECTION AND RESEARCH INSTITUTE, Guangzhou City (CN)

(72) Inventors: Bo YANG, Guangzhou City (CN); Guojia HUANG, Guangzhou City (CN); Maodong LI, Guangzhou City (CN); Yue LI, Guangzhou City (CN); Fang WEN, Guangzhou City (CN); Huachao GUO, Guangzhou City (CN); Wei ZHAI, Guangzhou City (CN); Shuanghong ZHANG, Guangzhou City (CN); Zhigang WANG, Guangzhou City (CN); Shiping LI, Guangzhou City (CN); Zhenling WU, Guangzhou City (CN); Yingyi HE, Guangzhou City (CN)

(21) Appl. No.: 16/458,764

(22) Filed: Jul. 1, 2019

(30) Foreign Application Priority Data
Jul. 6, 2018 (CN) 201810738611.4

(51) Int. Cl. C01B 32/192; C01G 49/10; C07C 275/02

(52) U.S. Cl. CPC C01B 32/192; C07C 275/02; C01G 49/10

(57) ABSTRACT

An intercalation agent for rapid graphite exfoliation in mass production of high-quality graphene is provided, including a transition metal halide salt, a nitrogen source substance and an organic solvent, and the mass ratio of the transition metal halide salt, the nitrogen source substance and the organic solvent is (1-10):1:(2-10). The transition metal halide salt can form a eutectic with the nitrogen source substance or the organic solvent, and the melting point thereof is lower than that of each component, thereby lowering the reaction temperature, and the preparation cost and difficulty; and a hydrogen bond can also be formed between the nitrogen source substance and the organic solvent, thereby avoiding interlayer stacking of the prepared graphene, thus improving the exfoliation efficiency and the product quality.

### CROSS-REFERENCE TO RELATED APPLICATIONS

This application claims priority to Chinese Application No. CN 201810738611.4 having a filing date of Jul. 6, 2018, the entire contents of which are hereby incorporated by reference.

### FIELD OF TECHNOLOGY

The following relates to the field of graphene preparing technology, and in particular relates to an intercalation agent for rapid graphite exfoliation in mass production of high-quality graphene.

### BACKGROUND

Graphene has excellent properties such as high electron mobility, ultrahigh thermal conductivity, good mechanical properties, and remarkable room-temperature Hall effect, which enables graphite and graphene related materials to be widely used in battery electrode materials, semiconductor devices, transparent displays, sea-water desalination, hydrogen storage materials, aerospace, composite materials, etc. In view of the excellent properties of graphene materials and their potential application value, the research and application development of graphene continues to heat up at home and abroad. Researchers focus on trying different methods in different fields to prepare high-quality graphene materials on a large scale, and reducing graphene preparation costs by continuously optimizing and improving the graphene preparation process, so that their excellent properties can be applied more widely.

Recently, some progress has been made on how to prepare high-quality graphene materials, but how to achieve mass production of high-yield and high-quality graphene is still a difficulty in research. Ball milling is a mechanical exfoliation method. The preparation of graphene by ball milling has attracted widespread attention from researchers due to its simple production process, low production cost, high yield and good quality.

The known art discloses a graphene preparation method, in which intercalation is carried out by using a composite intercalation agent first, and then exfoliation is achieved by ball milling shear to prepare graphene. In the preparation technology, a surfactant with a high boiling point is used in the preparation process of the composite intercalation agent, which is unfavorable for the subsequent drying treatment and thus affects the graphene quality; moreover, as the ultrasonic intercalation is carried out before ball milling, the preparation process is complicated, and the long-time ultrasonication not only affects the intercalation, but also causes the intercalation agent to fall off from the space between the layers, which is not conducive to the exfoliation of graphene, resulting in a low yield and low quality of the prepared graphene.

The known art also discloses a method for preparing graphene, in which intercalation is also carried out before ball milling, and an acid having strong oxidizing property and another oxidizing agent are used as an intercalation agent, and an expansion agent used therein is also an oxidizing substance. This method is essentially no different from the preparation of graphene by redox, does not reduce the defect degree of graphene, but also is more complicated than the redox preparation method.

According to the above description, an intercalation agent for preparing graphene by ball milling at present mainly adopts an acid having strong oxidizing property or other oxidizing agent, or a surfactant with a high boiling point is used in the preparation process, resulting in a low yield and low quality of the prepared graphene.

### SUMMARY

An aspect relates to an intercalation agent for rapid graphite exfoliation in mass production of high-quality graphene, with the advantages of environmental-friendliness and high exfoliation efficiency.

The embodiments of the present invention is achieved by the technical solution: an intercalation agent for rapid graphite exfoliation in mass production of high-quality graphene, including a transition metal halide salt, a nitrogen source substance and an organic solvent, wherein the mass ratio of the transition metal halide salt, the nitrogen source substance and the organic solvent is (1-10):1:(2-10).

Compared with the known art, the intercalation agent of embodiments of the present invention is a combination of the transition metal halide salt, the nitrogen source substance and the organic solvent, wherein as an electron-accepting intercalator, the transition metal halide salt accepts π electrons between the graphite layers during the intercalation process, and becomes negative ions and enters the space between the graphite layers; under the action of nitrogen source, the edges of graphite can be doped with nitrogen to form functional groups such as pyrrole and pyridine, so that the edges of the graphite layers turn up, thereby facilitating inserting the intercalation agent and the expansion agent between the layers to achieve interlayer exfoliation of graphite; and the organic solvent achieves a buffering effect in the ball milling process, thereby avoiding damage to the graphene structure by the solid-phase ball milling; moreover, it can disperse and stabilize the graphene obtained by exfoliation to prevent re-stacking of the graphene. They achieve a synergistic effect, wherein the transition metal halide salt can form a eutectic with the nitrogen source substance or the organic solvent, and the melting point thereof is lower than that of each component, and the mixed intercalation agent is even liquid at room temperature, and is inserted into graphite to form the graphite intercalation compound, thereby lowering the reaction temperature, and the preparation cost and difficulty; and a hydrogen bond can also be formed between the nitrogen source substance and the organic solvent, so that the bonding structure of the nitrogen source substance and the organic solvent is present stably between the graphene layers, thereby avoiding interlayer stacking of the prepared graphene, thus improving the exfoliation efficiency and the product quality. In addition, the intercalation agent of embodiments of the present invention does not undergo a chemical reaction during the ball milling process, and the intercalation agent and the graphite intercalation compound can be separated by centrifugation, and the separated intercalation agent can be recycled, which is energy-saving and environmentally friendly.

Further, the transition metal halide salt is any one or more of manganese chloride, chromium chloride, copper chloride, nickel chloride, ferrous bromide, ferric bromide, ferric chloride, and ferric chloride hexahydrate.

Further, the nitrogen source substance is any one or more of urea, dicyandiamide and melamine.

Further, the organic solvent is any one or more of ethanol, ethylene glycol, isopropanol, 1,2-propanediol, glycerol, formic acid, acetic acid, methyl acetate, ethyl acetate and ethyl formate.

Further, the transition metal halide salt is ferric chloride hexahydrate, the nitrogen source substance is urea, and the organic solvent is ethylene glycol.

Further, the mass ratio of ferric chloride hexahydrate, urea and ethylene glycol is 10:1:2.

Further, the transition metal halide salt is ferric chloride, the nitrogen source substance is urea, and the organic solvent is isopropanol.

Further, the mass ratio of ferric chloride, urea and isopropanol is 1:1:2.

Further, the transition metal halide salt is copper chloride, the nitrogen source substance is urea, and the organic solvent is ethanol.

Further, the mass ratio of copper chloride, urea and ethanol is 1:1:10.

For the sake of better understanding and implementation, embodiments of the present invention are described in detail below in conjunction with the accompanying drawings.

### BRIEF DESCRIPTION Some of the embodiments will be described in detail, with reference to the following figures, wherein like designations denote like members, wherein:

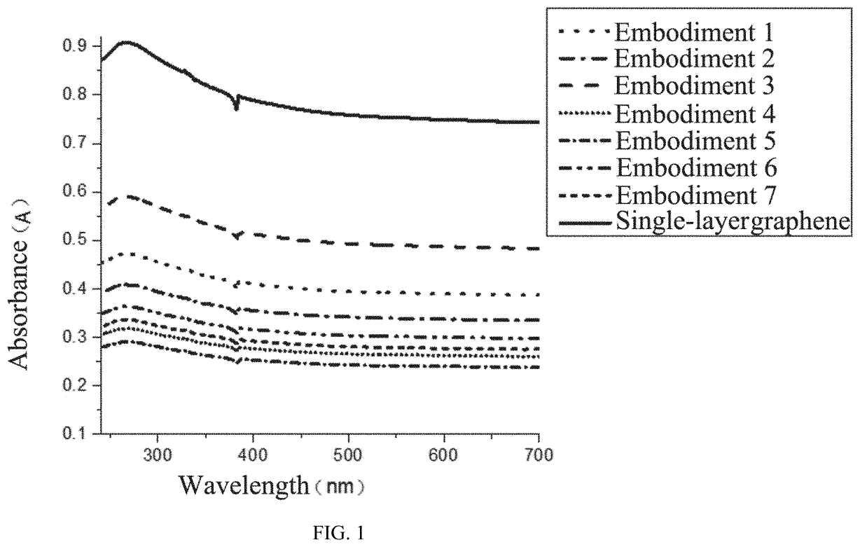

FIG. 1 illustrates ultraviolet absorbances of graphene dispersions of Embodiments 1 to 7 and a comparative single-layer graphene dispersion;

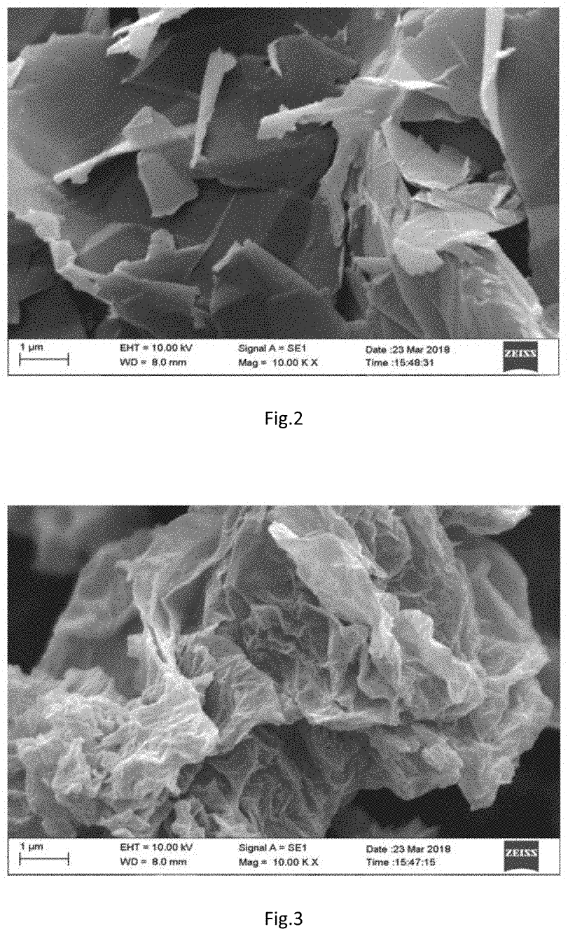

FIG. 2 shows a scanning electron microscope image of expanded graphite in Embodiment 1; and

FIG. 3 shows a scanning electron microscope image of a graphene product in Embodiment 1.

### DETAILED DESCRIPTION

In view of the problems in the preparation of graphene by the ball milling method at present that defects are serious and an organic solvent with a high boiling point is difficult to remove, in embodiments of the present invention, the intercalation agent is changed to a transition metal halide salt and a nitrogen source substance without strong oxidizing property and an organic solvent that is easy to remove, which are used in combination as an environmentally friendly intercalation agent, which has an intercalating effect during ball milling and achieves partial exfoliation, so that the complicated process of ball milling after intercalation can be avoided; then simple cleaning is carried out to remove the intercalation agent on the surface of the intercalation compound; after filtering, the intercalation compound is further subjected to expansion treatment to obtain high-quality and high-yield graphene. The operation is simple and the production cost is low, which are favorable for promoting the mass production of graphene. Moreover, the corrosion effect of a strong oxidant on the a jar mill is avoided, thereby achieving the combination of the intercalation and the ball-milling exfoliation process, which not only improves the exfoliation efficiency, but also can maintain the integrity of the graphene crystal structure, thus expanding the applications of graphene in the fields of energy storage materials, biomaterials and the like.

The intercalation agent for rapid graphite exfoliation in mass production of high-quality graphene in embodiments of the present invention includes a transition metal halide salt, a nitrogen source substance and an organic solvent, wherein the mass ratio of the transition metal halide salt, the nitrogen source substance and the organic solvent is (1-10):1:(2-10).

Specifically, the transition metal halide salt can be any one or more of manganese chloride, chromium chloride, copper chloride, nickel chloride, ferrous bromide, ferric bromide, ferric chloride, and ferric chloride hexahydrate. The nitrogen source substance can be any one or more of urea, dicyandiamide and melamine. The organic solvent can be any one or more of ethanol, ethylene glycol, isopropanol, 1,2-propanediol, glycerol, formic acid, acetic acid, methyl acetate, ethyl acetate and ethyl formate.

Preferably, the intercalation agent includes ferric chloride hexahydrate, urea and ethylene glycol, the mass ratio of which is 10:1:2. Further preferably, the intercalation agent includes ferric chloride, urea, isopropanol, the mass ratio of which is 1:1:2. Still preferably, the intercalation agent includes copper chloride, urea, ethanol, the mass ratio of which is 1:1:10.

A method for preparing graphite using the intercalation agent of embodiments of the present invention to exfoliate graphene includes the following steps:

S1: mixing a transition metal halide salt, a nitrogen source substance and an organic solvent to prepare an intercalation agent;
S2: mixing the intercalation agent obtained in the step S1 with graphite, carrying out ball milling, and then performing centrifugation to obtain a graphite intercalation compound;
S3: washing and filtering the graphite intercalation compound obtained in the step S2, adding an expansion agent, and carrying out ultrasonic agitation to obtain a graphene dispersion; and
S4: washing, filtering and drying the graphene dispersion obtained in the step S3 to obtain graphene powder.

Specifically, in the step S2, the mass ratio of the intercalation agent to the graphite is (40-200):1. Zirconia balls are used in the ball milling process, and the volume ratio of the intercalation agent to the zirconia balls is (1-3): 1, and the total volume of the intercalation agent and the zirconia balls accounts for 25-60% of the volume of the jar mill. The ball milling speed is 200-700 rpm, and the ball milling time is 2-48 h. The speed during centrifugation is 8000-10000 rpm. The graphite is any one or more of expanded graphite, expandable graphite, natural flake graphite and graphite powder.

In the step S3, the expansion agent is any one or more of hydrogen peroxide, sodium borohydride and ammonium bicarbonate. The agitating speed is 200-600 r/min.

In the step S4, the drying process is carried out by using a vacuum oven for drying at a temperature of 60-80° C. for 12-24 hours.

Further description is provided below in conjunction with specific embodiments.

### Embodiment 1

The intercalation agent for rapid graphite exfoliation in mass production of high-quality graphene in the embodiment includes a transition metal halide salt, a nitrogen source substance and an organic solvent, the mass ratio of which is 1:1:2, wherein the transition metal halide salt is ferric chloride, the nitrogen source substance is urea, and the organic solvent is isopropanol.

The method for preparing graphene using the intercalation agent to exfoliate graphite specifically includes the following steps: 1 g of expanded graphite was poured into a dry jar mill, then 10 g of ferric chloride, 10 g of urea, 20 g of isopropanol were mixed uniformly and poured into the jar mill so that zirconia beads were submerged therein; the jar mill was fixed symmetrically to a planetary ball mill, the rotating speed was adjusted to 700 rpm, and ball milling was carried out continuously for 48 hours; after the ball milling, the graphite intercalation compound was separated from the intercalation agent by high-speed centrifugation at 10,000 rpm, and then the intercalation compound was simply washed with water to remove the intercalation agent on the surface of the graphite intercalation compound; after filtration, a filter cake was placed into a flask, 50 ml of 5% hydrogen peroxide was added therein, water-bath sonication was carried out for 20 min, and the solution was stirred for 2 h to decompose hydrogen peroxide; then the solution was filtered, washed 3 times, and dried at 80° C. for 12 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

### Embodiment 2

The intercalation agent for rapid graphite exfoliation in mass production of high-quality graphene in the embodiment includes a transition metal halide salt, a nitrogen source substance and an organic solvent, the mass ratio of which is 1:1:10, wherein the transition metal halide salt is copper chloride, the nitrogen source substance is urea, and the organic solvent is ethanol.

The method for preparing graphene using the intercalation agent to exfoliate graphite specifically includes the following steps: 1 g of 300-mesh flake graphite was poured into a dry jar mill, then 5 g of copper chloride, 5 g of urea, 50 g of absolute ethanol were mixed uniformly and poured into the jar mill so that zirconia beads were submerged therein. The jar mill was fixed symmetrically to a planetary ball mill, the rotating speed was adjusted to 500 rpm, and ball milling was carried out continuously for 48 hours; after the ball milling, the graphite intercalation compound was separated from the intercalation agent by high-speed centrifugation at 10,000 rpm, and then the intercalation compound was simply washed with water to remove the intercalation agent on the surface of the graphite intercalation compound; after filtration, a filter cake was placed into a flask, 50 ml of 5% sodium borohydride solution was added therein, water bath sonication was carried out for 20 min, then the pH of the solution was adjusted to make the solution acidic, and stirring was carried out for 2 h to decompose sodium borohydride; then the solution was filtered, washed 3 times, and dried at 60° C. for 24 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

### Embodiment 3

The intercalation agent for rapid graphite exfoliation in mass production of high-quality graphene in the embodiment includes a transition metal halide salt, a nitrogen source substance and an organic solvent, the mass ratio of which is 10:1:2, wherein the transition metal halide salt is ferric chloride hexahydrate, the nitrogen source substance is urea, and the organic solvent is ethylene glycol.

The method for preparing graphene using the intercalation agent to exfoliate graphite specifically includes the following steps: 1 g of graphite powder was poured into a dry jar mill, then 50 g of ferric chloride hexahydrate, 5 g of urea, 10 g of ethylene glycol were mixed uniformly and poured into the jar mill so that zirconia beads were submerged therein; the jar mill was fixed symmetrically to a planetary ball mill, the rotating speed was adjusted to 400 rpm, and ball milling was carried out continuously for 48 hours; after the ball milling, the graphite intercalation compound was separated from the intercalation agent by high-speed centrifugation at 10,000 rpm, and then the intercalation compound was simply washed with water to remove the intercalation agent on the surface of the graphite intercalation compound; after filtration, a filter cake was placed into a flask, 50 ml of 5% hydrogen peroxide was added therein, water-bath sonication was carried out for 20 min, and the solution was stirred for 2 h to decompose hydrogen peroxide; then the solution was filtered, washed 3 times, and dried at 80° C. for 12 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

### Embodiment 4

The intercalation agent for rapid graphite exfoliation in mass production of high-quality graphene in the embodiment includes a transition metal halide salt, a nitrogen source substance and an organic solvent, the mass ratio of which is 4:1:6, wherein the transition metal halide salt is chromium chloride, the nitrogen source substance is melamine, and the organic solvent is a mixture of ethylene glycol and glycerin.

The method for preparing graphene using the intercalation agent to exfoliate graphite specifically includes the following steps: 1 g of graphite powder was poured into a dry jar mill, then 20 g of chromium chloride, 5 g of melamine, 20 g of ethylene glycol, and 10 g of glycerin were mixed uniformly and poured into the jar mill so that zirconia beads were submerged therein; the jar mill was fixed symmetrically to a planetary ball mill, the rotating speed was adjusted to 600 rpm, and ball milling was carried out continuously for 48 hours; after the ball milling, the graphite intercalation compound was separated from the intercalation agent by high-speed centrifugation at 10,000 rpm, and then the intercalation compound was simply washed with hot water to remove the intercalation agent on the surface of the graphite intercalation compound; after filtration, a filter cake was placed into a flask, 50 ml of 5% ammonium bicarbonate was added therein, water-bath sonication was carried out at 75° C. for 20 min, and the solution was stirred for 2 h; then the solution was filtered, washed 3 times, and dried at 80° C. for 12 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

### Embodiment 5

The intercalation agent for rapid graphite exfoliation in mass production of high-quality graphene in the embodiment includes a transition metal halide salt, a nitrogen source substance and an organic solvent, the mass ratio of which is 5:1:10, wherein the transition metal halide salt is nickel chloride, the nitrogen source substance is dicyandiamide, and the organic solvent is methyl acetate.

The method for preparing graphene using the intercalation agent to exfoliate graphite specifically includes the following steps: 1 g of expandable graphite was poured into a dry jar mill, then 25 g of nickel chloride, 5 g of dicyandiamide, 50 g of methyl acetate were mixed uniformly and poured into the jar mill so that zirconia beads were submerged therein; the jar mill was fixed symmetrically to a planetary ball mill, the rotating speed was adjusted to 400 rpm, and ball milling was carried out continuously for 48 hours; after the ball milling, the graphite intercalation compound was separated from the intercalation agent by high-speed centrifugation at 10,000 rpm, and then the intercalation compound was simply washed with hot water to remove the intercalation agent on the surface of the graphite intercalation compound; 50 ml of 5% sodium borohydride solution was added therein, water bath sonication was carried out for 20 min, then the pH of the solution was adjusted to make the solution acidic, and stirring was carried out for 2 h to decompose sodium borohydride; then the solution was filtered, washed 3 times, and dried at 80° C. for 12 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

### Embodiment 6

The intercalation agent for rapid graphite exfoliation in mass production of high-quality graphene in the embodiment includes a transition metal halide salt, a nitrogen source substance and an organic solvent, the mass ratio of which is 1:1:5, wherein the transition metal halide salt is ferrous bromide, the nitrogen source substance is urea, and the organic solvent is ethyl acetate.

The method for preparing graphene using the intercalation agent to exfoliate graphite specifically includes the following steps: 1 g of expanded graphite, 10 g of ferrous bromide, 10 g of urea, and 50 g of ethyl acetate were mixed uniformly and poured into the jar mill so that zirconia beads were submerged therein; the jar mill was fixed symmetrically to a planetary ball mill, the rotating speed was adjusted to 500 rpm, and ball milling was carried out continuously for 48 hours; after the ball milling, the graphite intercalation compound was separated from the intercalation agent by high-speed centrifugation at 10,000 rpm, and then the intercalation compound was simply washed with ethanol to remove the intercalation agent on the surface of the graphite intercalation compound; after filtration, a filter cake was placed into a flask, 50 ml of 5% ammonium bicarbonate was added therein, water-bath sonication was carried out at 75° C. for 20 min, and the solution was stirred for 2 h; then the solution was filtered, washed 3 times, and dried at 60° C. for 24 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

### Embodiment 7

The intercalation agent for rapid graphite exfoliation in mass production of high-quality graphene in the embodiment includes a transition metal halide salt, a nitrogen source substance and an organic solvent, the mass ratio of which is 10:1:10, wherein the transition metal halide salt is a mixture of ferric chloride and nickel chloride, the nitrogen source substance is urea, and the organic solvent is ethanol.

The method for preparing graphene using the intercalation agent to exfoliate graphite specifically includes the following steps: 1 g of 300-mesh flake graphite was poured into a dry jar mill, then 25 g of ferric chloride, 25 g of nickel chloride, 5 g of urea and 50 g of ethanol were mixed uniformly and poured into the jar mill so that zirconia beads were submerged therein; the jar mill was fixed symmetrically to a planetary ball mill, the rotating speed was adjusted to 600 rpm, and ball milling was carried out continuously for 48 hours; after the ball milling, the graphite intercalation compound was separated from the intercalation agent by high-speed centrifugation at 10,000 rpm, and then the intercalation compound was simply washed with water to remove the intercalation agent on the surface of the graphite intercalation compound; after filtration, a filter cake was placed into a flask, 50 ml of 5% hydrogen peroxide was added therein, water-bath sonication was carried out for 20 min, and the solution was stirred for 2 h to decompose hydrogen peroxide; then the solution was filtered, washed 3 times, and dried at 80° C. for 12 h to obtain graphene powder.

0.06 g of the obtained graphene powder was dispersed in a 50 mg/ml aqueous urea solution to prepare a graphene dispersion with a concentration of 0.024 mg/ml. Then, moderate sonication was carried out for 15 min, and the absorbance of the graphene dispersion at 270 nm was measured by ultraviolet-visible spectroscopy and compared with the absorbance of a prepared single-layer graphene dispersion with the same concentration at 270 nm to measure the concentration of graphene and calculate the yield of graphene.

The single-layer graphene for comparison was purchased from Nanjing XFNANO Materials Tech Co., Ltd, and the model of the graphene was XF001W.

The absorbance of the graphene dispersions of Embodiments 1 to 7 and the absorbance of the single-layer graphene dispersion for comparison were detected, and the results are shown in FIG. 1. The graphene yields of Embodiments 1 to 7 were calculated, and the results are shown in Table 1 below.

TABLE 1
Summary of reagent types and graphene
yields of Embodiments 1-7

| | | | | Graph- |
|---|---|---|---|---|
| Embodi- | | | Expansion | ene |
| ment | intercalation agent | Graphite | agent | yield |
| 1 | Ferric chloride, urea, | Expanded | Hydrogen | 52% |
| | isopropanol | graphite | peroxide | |
| 2 | Copper chloride, urea, | 300-mesh | Sodium | 45% |
| | absolute ethanol | flake | borohydride | |
| | | graphite | solution | |
| 3 | Ferric chloride | Graphite | Hydrogen | 65% |
| | hexahydrate, urea, | powder | peroxide | |
| | ethylene glycol | | | |
| 4 | Chromium chloride, | Graphite | Ammonium | 35% |
| | melamine, ethylene | powder | bicarbonate | |
| | glycol, glycerin | | solution | |
| 5 | Nickel chloride, | Expandable | Sodium | 32% |
| | dicyandiamide, | graphite | borohydride | |
| | methyl acetate | | solution | |
| 6 | Ferrous bromide, urea, | Expanded | Ammonium | 40% |
| | ethyl acetate | graphite | bicarbonate | |
| | | | solution | |
| 7 | Ferric chloride, nickel | 300-mesh | Hydrogen | 37% |
| | chloride, urea, ethanol | flake | peroxide | |
| | | graphite | | |

It can be seen from FIG. 1 that the final ball-milled graphene yields in the different embodiments are different, wherein the yield in Embodiment 3 is the highest, which is attributed to the good synergistic effect of ferric chloride hexahydrate, urea and ethylene glycol.

In addition, the morphology of the expanded graphite and the graphene product in Embodiment 1 was tested to obtain a scanning electron microscope (SEM) image, as shown in FIGS. 2 and 3, wherein at the same magnification (10K times), the surface of expanded graphite (as shown in FIG. 2) is smooth and exhibits irregular-size flake structures, while the ball-milled graphene (shown in FIG. 3) has flexibility and obvious pleats, with some graphene layers folded and stacked on each other.

Compared with the known art, embodiments of the present invention use the synergistic effect among the transition metal halide salt, the nitrogen source substance and the organic solvent to prepare the intercalation agent, wherein the transition metal halide salt can form a eutectic with the nitrogen source substance or the organic solvent, and the melting point thereof is lower than that of each component, and the mixed intercalation agent is even liquid at room temperature, and is inserted into graphite to form the graphite intercalation compound, thereby lowering the reaction temperature, and the preparation cost and difficulty; and a hydrogen bond can also be formed between the nitrogen source substance and the organic solvent, so that the bonding structure of the nitrogen source substance and the organic solvent is present stably between the graphene layers, thereby avoiding interlayer stacking of the prepared graphene, thus improving the exfoliation efficiency and the product quality. In addition, the intercalation agent of embodiments of the present invention does not undergo a chemical reaction during the ball milling process, and the intercalation agent and the graphite intercalation compound can be separated by centrifugation, and the separated intercalation agent can be recycled, which is energy-saving and environmentally friendly.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

What is claimed:

1. An intercalation agent for graphite exfoliation in mass production of graphene, including a transition metal halide salt, a nitrogen source substance and an organic solvent, and the mass ratio of the transition metal halide salt, the nitrogen source substance and the organic solvent is (1-10):1:(2-10).

2. The intercalation agent of claim 1, wherein the transition metal halide salt is any one or more of manganese chloride, chromium chloride, copper chloride, nickel chloride, ferrous bromide, ferric bromide, ferric chloride, and ferric chloride hexahydrate.

3. The intercalation agent of claim 1, wherein the nitrogen source substance is any one or more of urea, dicyandiamide and melamine.

4. The intercalation agent of claim 1, wherein the organic solvent is any one or more of ethanol, ethylene glycol, isopropanol, 1,2-propanediol, glycerol, formic acid, acetic acid, methyl acetate, ethyl acetate and ethyl formate.

5. The intercalation agent claim 1, wherein the transition metal halide salt is ferric chloride hexahydrate, the nitrogen source substance is urea, and the organic solvent is ethylene glycol.

6. The intercalation agent of claim 5, wherein the mass ratio of ferric chloride hexahydrate, urea and ethylene glycol is 10:1:2.

7. The intercalation agent of claim 1, wherein the transition metal halide salt is ferric chloride, the nitrogen source substance is urea, and the organic solvent is isopropanol.

8. The intercalation agent of claim 7, wherein the mass ratio of ferric chloride, urea and isopropanol is 1:1:2.

9. The intercalation agent of claim 1, wherein the transition metal halide salt is copper chloride, the nitrogen source substance is urea, and the organic solvent is ethanol.

10. The intercalation agent of claim 9, wherein the mass ratio of copper chloride, urea and ethanol is 1:1:10.
